SEQUENCE LISTING (32 sequences)

SEQ ID NO: 1 - protein, 7 aa, Artificial
  Synthetic Construct
AWLDSGV

SEQ ID NO: 2 - protein, 4 aa, Artificial
  Synthetic Construct
FSDY

SEQ ID NO: 3 - protein, 6 aa, Artificial
  Synthetic Construct
GFSDYK

SEQ ID NO: 4 - protein, 6 aa, Artificial
  Synthetic Construct
  misc_feature (2)..(5): Xaa can be any naturally occurring amino acid except cysteine
GXXXXK

SEQ ID NO: 5 - protein, 6 aa, Artificial
  Synthetic Construct
  MISC_FEATURE (2)..(3): Xaa can be any naturally occurring amino acid except cysteine, aspartate and glutamate
  MISC_FEATURE (5)..(5): Xaa can be any naturally occurring amino acid except cysteine
GXXDXK

SEQ ID NO: 6 - protein, 6 aa, Artificial
  Synthetic Construct
  MISC_FEATURE (2)..(2): Xaa can be any naturally occurring amino acid except cysteine, aspartate and glutamate
  MISC_FEATURE (3)..(3): Xaa can be any naturally occurring amino acid except cysteine
  MISC_FEATURE (5)..(5): Xaa can be any naturally occurring amino acid except cysteine, aspartate and glutamate
GXXDXK

SEQ ID NO: 7 - protein, 6 aa, Artificial
  Synthetic Construct
  MISC_FEATURE (2)..(2): Xaa can be any naturally occurring amino acid except cysteine
  MISC_FEATURE (3)..(3): Xaa can be any naturally occurring amino acid except cysteine, aspartate and glutamate
  MISC_FEATURE (5)..(5): Xaa can be any naturally occurring amino acid except cysteine, aspartate and glutamate
GXXDXK

SEQ ID NO: 8 - protein, 4 aa, Artificial
  Synthetic Construct
DRRL

SEQ ID NO: 9 - protein, 4 aa, Artificial
  Synthetic Construct
RHTR

SEQ ID NO: 10 - protein, 6 aa, Artificial
  Synthetic Construct
  misc_feature (2)..(3): Xaa can be any naturally occurring amino acid
  misc_feature (5)..(5): Xaa can be any naturally occurring amino acid
GXXDXK

SEQ ID NO: 11 - protein, 6 aa, Artificial
  Synthetic Construct
  misc_feature (2)..(3): Xaa can be any naturally occurring amino acid
  misc_feature (5)..(5): Xaa can be any naturally occurring amino acid
GXXEXK

SEQ ID NO: 12 - protein, 6 aa, Artificial
  Synthetic Construct
GFSEYK

SEQ ID NO: 13 - protein, 6 aa, Artificial
  Synthetic Construct
GFSDYQ

SEQ ID NO: 14 - protein, 9 aa, Artificial
  Synthetic Construct
DHLSDTSTQ

SEQ ID NO: 15 - protein, 7 aa, Artificial
  Synthetic Construct
LELDSRQ

SEQ ID NO: 16 - protein, 8 aa, Artificial
  Synthetic Construct
AWLDSGVQ

SEQ ID NO: 17 - protein, 14 aa, Artificial
  Synthetic Construct
RRHTRSAEDDSERQ

SEQ ID NO: 18 - protein, 8 aa, Artificial
  Synthetic Construct
LMMDFRGQ

SEQ ID NO: 19 - protein, 8 aa, Artificial
  Synthetic Construct
SAEDNSPQ

SEQ ID NO: 20 - protein, 8 aa, Artificial
  Synthetic Construct
RSEDAGFQ

SEQ ID NO: 21 - protein, 8 aa, Artificial
  Synthetic Construct
NGYDVYHQ

SEQ ID NO: 22 - protein, 14 aa, Artificial
  Synthetic Construct
RRHTQSAEDDSERQ

SEQ ID NO: 23 - protein, 8 aa, Artificial
  Synthetic Construct
GSSDAAEQ

SEQ ID NO: 24 - protein, 8 aa, Artificial
  Synthetic Construct
RRDDSSEQ

SEQ ID NO: 25 - protein, 8 aa, Artificial
  Synthetic Construct
IPSDFEGQ

SEQ ID NO: 26 - protein, 9 aa, Artificial
  Synthetic Construct
GPQRDSQAQ

SEQ ID NO: 27 - protein, 8 aa, Artificial
  Synthetic Construct
THLDTKKQ

SEQ ID NO: 28 - protein, 8 aa, Artificial
  Synthetic Construct
GSNDIMGQ

SEQ ID NO: 29 - protein, 8 aa, Artificial
  Synthetic Construct
RGLDNEIQ

SEQ ID NO: 30 - protein, 8 aa, Artificial
  Synthetic Construct
NEMDSFNQ

SEQ ID NO: 31 - protein, 7 aa, Artificial
  Synthetic Construct
SSDSGSQ

SEQ ID NO: 32 - protein, 6 aa, Artificial
  Synthetic Construct
GFRDWK

(19) United States
(12) Patent Application Publication
(10) Pub. No.: US 2006/0292657 A1
(43) Pub. Date: Dec. 28, 2006

(54) Phex substrates and methods using same

(76) Inventors: Guy Boileau, Quebec (CA); Adriana Karaoglanovic Carmona, Sao Paulo (BR); Marcelo Campos, Sao Paulo (BR); Maria Aparecida Juliano, Sao Paulo (BR); Luiz Juliano, Sao Paulo (BR)

Correspondence Address: Choate Hall & Stewart;Patent Group, Two International Place, Boston, MA, 02110, US

(21) Appl. No.: 10/549,796

(22) PCT Filed: Mar. 25, 2004
(86) PCT No.: PCT/CA04/00453

§ 371 Date: Jun. 22, 2006

Related U.S. Application Data
(60) Provisional application No. 60/457,296, filed on Mar. 26, 2003.

(51) Int. Cl. C12Q 1/37; C07K 7/06

(52) U.S. Cl. 435/23; 530/329; 530/330

(57) ABSTRACT

A fluorogenic PHEX substrate comprising a peptide unit; a fluorophore unit capable of conferring fluorescence on said substrate attached to an amino acid residue at a first end of the peptide unit; and a quencher unit capable of providing intrarnolecular quenching of said fluorescence attached to an amino acid residue at a second end of the peptide unit; the peptide unit having at least 6 amino acids residues including a sequence P₂-P₁-P₁-P₂, of 4 amino acid residues at positions P₂, P₁, P₁, and P₂ of the peptide unit, respectively; the amino acid residue at position P₂ being any amino acid residue; the amino acid residue at position P, being any amino acid residue except an isoleucine, a valine, or a histidine residue; the amino acid residue at position P₁, being an acidic amino acid residue selected from the group consisting of a glutamic acid residue and an aspartic acid residue, and being located at least 2 amino acid residues distal to both the fluorophore and the quencher units; the amino acid residue at position P₂, being any amino acid residue except a leucine, a proline or a glycine residue, with the proviso that said peptide unit does not have the sequence as set forth in SEQ ID NO: 1. Methods of using the peptide sequence unit to identify PHEX modulators and for detecting PHEX in a sample.

### FIELD OF THE INVENTION

The present invention relates to substrates of PHEX, the enzyme encoded by the PHEX gene (phosphate-regulating gene with homologies to endopeptidases on the X chromosome) and methods of using same. More specifically, the present invention relates to peptidic PHEX enzyme substrates and their use in methods to determine the PHEX enzyme activity and in assays amenable to high throughput screening for PHEX modulators.

### BACKGROUND OF THE INVENTION

PHEX was first identified by positional cloning as the gene that is mutated in patients with X-linked hypophosphatemia (XLH) (1). XLH is the most prevalent form of inherited rickets in humans and is characterized by growth retardation, rachitic and osteomalacic bone disease, hypophosphatemia, and renal defects in phosphate reabsortion and vitamin D metabolism (2). Much of the knowledge about XLH has been obtained from studies of the Hyp mouse, which harbors a large deletion of the PHEX gene (3) and has been used as an animal model of the human disease (4), confirming that PHEX plays an important role in the control of phosphate homeostasis and skeletal mineralization. However, the mechanism(s) by which PHEX regulates these physiological processes is still unknown.

The product of the PHEX gene shows striking homologies to members of the M13 family of zinc metalloendopeptidase. These enzymes are type 11 integral membrane glycoproteins (5) and include Neprilysin (NEP, neutral endopeptidase 24.11) (6), Endothelin-Converting Enzymes (ECE-1 and ECE-2) (7), Kell blood group protein (8), ECE-like enzyme/distress induced neuronal endopeptidase (ECELL) (9), soluble endopeptidase/NEP-like enzyme 1/neprilysin 2 (NL1) (10) and membrane metallo-endopeptidase-like 2 (MMEL2) (11). The use of specific inhibitors has demonstrated the involvement of NEP and ECE-1 and ECE-2 in regulating the amount and hence activity of several bioactive peptides (12,13). Sequence similarity of PHEX to other members of M13 family suggests a similar role for PHEX.

The major sites of PHEX expression are bone (3,16-17) and teeth (17), but PHEX mRNA was also detected in ovary (18), fetal lung (3) and the parathyroid gland (19). PHEX expression is notably absent from kidney (3), suggesting that it may regulate renal phosphate reabsorption by controlling the activity of a circulating factor. Consistent with this hypothesis was the demonstration that inhibition of Na-dependent phosphate transport in cultured renal cells can be achieved by a factor in conditioned medium from cultured osteoblasts derived from Hyp mice (20).

Phosphaturic activity(ies) have also been found in tumors from patients with tumor-induced osteomalacia (TIO, also known as oncogenic hypophosphatemic osteomalacia), an acquired renal phosphate wasting disorder with the phenotypic features of XLH (2). The term “phosphatonin” was originally designated to depict these phosphaturic tumor factor(s) (21) and although the exact nature of “phosphatonin” remains to be determined, several candidates have been proposed (for a review see 22). It has been shown that mutations in the FGF-23gene that encodes a novel growth factor, fibroblast growth factor-23(FGF-23) is responsible for Autosomal Dominant Hypophosphatemic Rickets (ADHR), an inherited disorder that resembles XLH and TIO (23). Moreover, it was demonstrated that overexpression of FGF-23in animal models elicits renal phosphate wasting, a reduction in serum phosphate levels and osteomalacia (24). Of interest was the finding that FGF-23is overexpressed in tumors from patients with TIO (25). In addition to FGF-23, other proteins such as Frizzled-related protein 4 (FRP4) (25) and MEPE (matrix extracellular phosphoglycoprotein) (26) are overexpressed in TIO tumors. However, the phosphaturic activity of FRP-4 and MEPE in animal models remains to be determined. The PHEX( enzyme was proposed as a phosphatonin-processing factor in a manner similar to that of other membrane-bound peptidases like NEP and ACE. (Kumar, Bone 27(3) (2000) 333-8). 7 From these considerations, several PHEX substrate candidates such as PTH (20), PTHrP(107-139) (13), FGF-23and MEPE have been proposed, but no physiologically relevant PHEX substrate has yet been identified. In addition, there is much conflicting data in the literature about the hydrolytic activity of PHEX (14,15,28,27,28). Lipman at al (20) reported that PTH(1-34) was cleaved by a PHEX preparation. However, Guo et al were unable to reproduce these results (27), and suggested that this was probably due to proteolytic impurities in the Lipman membrane preparation. Instead, these authors claimed that the NEP substrate, [D-Ala2,Leu5 Enkephalin was a PHEX substrate. The international application WO 00/50580 published in August 2000 in the name of Crine et al. provides for an enzymatic assay for PHEX, whereupon the peptide PTHrP(107-139) is cleaved in three fragments by pure soluble form of PHEX (secPHEX) with the cleavage products determined by HPLC. The multiple cleavage of PTHrP(107-139) combined to the chromatographic step make this assay not easily amenable to high throughput format to identify new PHEX inhibitors or for the determination of PHEX in biological fluids like serum or cell growth media. In addition, when various segments of PTHrP(107-139) comprising one cleavage site are prepared and tested for enzymatic activity with PHEX, the cleavage rate is much lower than with the full peptide. In this context there is a need for a high throughput assay where the substrate peptide is rapidly cleaved by PHEX with sensitive detection limits.

Toward these goals, extensive libraries were designed to identify new substrates using internally quenched fluorogenic peptides (IQFPs) containing the groups Abz (ortho-amino benzoic acid) as fluorescent donor, and EDDnp or Dnp as fluorescent acceptor (quencher). In these libraries, when EDDnp was used as quencher, it was attached to a glutamine residue (i.e. glutaminyl-[N-(2,4-dinitrophenyl) ethylenediamine]), and when Dnp was used as quencher, it was attached to a lysine residue (i.e. lysil-2,4-dinitrophenyl). It was discovered that in contrast to reports in the literature, PHEX did not show a Neprylisin-like activity but exhibited a strict requirement for acidic amino acid residues (Asp or Glu) in S₁′ subsite with a strong preference for Asp. The Boileau et aL disclosures (14 and WO 02/15918 published in February 2002) further identified that the DT or DS pair was cleaved efficiently by PHEX at the N-terminal side of aspartic residue.

An object of the present invention is therefore the development of new more specific peptide motifs as well as new IQFPs that can be cleaved by the PHEX enzyme and assays using these peptide motifs and IQFPs.

### SUMMARY OF THE INVENTION

In accordance with the present invention, there are therefore provided PHEX substrates and assays using same including assays to determine the presence/concentration of PHEX in samples such as biological media and assays for identifying PHEX modulators and more specifically, PHEX inhibitors.

More specifically, in accordance with the present invention, there is provided a fluorogenic PHEX substrate comprising a peptide unit; a fluorophore unit capable of conferring fluorescence on said substrate attached to an amino acid residue at a first end of the peptide unit; and a quencher unit capable of providing intramolecular quenching of said fluorescence attached to an amino acid residue at a second end of the peptide unit; the peptide unit having at least 6 amino acids residues including a sequence P₂-P₁-P₁′-P₂′ of 4 amino acid residues at positions P₂, P₁, P₁′ and P₂′ of the peptide unit, respectively; the amino acid residue at position P₂ being any amino acid residue; the amino acid residue at position P₁ being any amino acid residue except an isoleucine, a valine, or a histidine residue; the amino acid residue at position P₁′ being an acidic amino acid residue selected from the group consisting of a glutamic acid residue and an aspartic acid residue, and being located at least 2 amino acid residues distal to both the fluorophore and the quencher units; the amino acid residue at position P₂′ being any amino acid residue except a leucine, a proline or a glycine residue, with the proviso that said peptide unit does not have the sequence as set forth in SEQ ID NO:1.

There is also provided a method for identifying a PHEX modulator comprising contacting a candidate compound with PHEX in the presence of a PHEX substrate, said substrate including a peptide unit of at least 6 amino acids residues including a sequence P₂-P₁-P₁′-P₂′ of 4 amino acid residues at positions P₂, P₁, P₁′ and P₂′ of the peptide unit, respectively; the amino acid residue at position P₂ being any amino acid residue; the amino acid residue at position P₁ being any amino acid residue except an isoleucine, a valine, or a histidine residue; the amino acid residue at position P₁ being an acidic amino acid residue selected from the group consisting of a glutamic acid and an aspartic acid residue; the amino acid residue at position P₂′ being any amino acid residue except a leucine, a proline or a glycine residue, with the proviso that said peptide does not have the sequence as set forth in SEQ ID NO:1; detecting a product resulting of the PHEX enzymatic activity on said substrate, wherein a difference in the amount of said product detected in the presence of said candidate compound as compared to that in the absence thereof is an indication that said candidate compound modulates PHEX.

There is also provided a method for determining the presence and/or concentration of PHEX in a sample comprising contacting said sample with a PHEX peptide substrate, said substrate including a peptide unit of at least 6 amino acids residues including a sequence P₂-P₁-P₁′-P₂′ of 4 amino acid residues at positions P₂, P₁, P₁′ and P₂′ of the peptide unit, respectively; the amino acid residue at position P₂ being any amino acid residue; the amino acid residue at position P₁ being any amino acid residue except an isoleucine, a valine, or a histidine residue; the amino acid residue at position P₁′ being an acidic amino acid residue selected from the group consisting of a glutamic acid and an aspartic acid residue; the amino acid residue at position P₂′ being any amino acid residue except a leucine, a proline or a glycine residue, with the proviso that said peptide does not have the sequence as set forth in SEQ ID NO:1; assessing the presence and/or concentration of a product resulting of the PHEX enzymatic activity on said substrate; and wherein the presence and/or concentration of said product can be correlated to the presence/concentration of PHEX in the sample.

In specific embodiments, the above-described methods further comprise a method a fluorophore unit capable of conferring fluorescence on said substrate, said fluorophore unit being attached to an amino acid residue at a first end of the peptide unit; and a quencher unit capable of providing intramolecular quenching of said fluorescence, said quencher unit being attached to an amino acid residue at a second end of the peptide unit, wherein P₁′ is located at least 2 amino acid residues distal to both the fluorophore unit and the quencher unit and wherein the product is detected through a modulation of fluorescence.

In accordance with the present invention, there is also provided a fluorogenic PHEX substrate comprising a peptide unit; a fluorophore unit capable of conferring fluorescence on said substrate, said fluorophore being aftached to an amino acid residue at a first end of the peptide unit; and a quencher unit capable of providing intramolecular quenching of said fluorescence, said quencher being attached to an amino acid residue at a second end of the peptide unit; the peptide unit comprising the sequence P₃-P₂-P₁-P₁′-P₂′-P₃′ of amino acid residues at positions P₃, P₂, P₁, P₁′, P₂′, and P₃′ of the peptide unit, respectively; the amino acid residue at position P₂ being any amino acid residue; the amino acid residue at position P₁ being any amino acid residue except an isoleucine, a valine, or a histidine residue; the amino acid residue at position P₁′ being an acidic amino acid residue selected from the group consisting of a glutamic acid residue and an aspartic acid residue; the amino acid residue at position P₂′ being any amino acid residue except a leucine, a proline or a glycine residue, with the proviso that said peptide unit does not have the sequence as set forth in SEQ ID NO:1.

There is also provided a method for identifying a PHEX modulator comprising contacting a candidate compound with PHEX in the presence of this fluorogenic PHEX substrate comprising detecting a product resulting of the PHEX enzymatic activity on said substrate, wherein a difference in the amount of said product detected in the presence of said candidate compound as compared to that in the absence thereof is an indication that said candidate compound modulates PHEX. There is also provided a method for determining the presence and/or concentration of PHEX in a sample comprising contacting said sample with the fluorogenic PHEX substrate, detecting a product resulting of the PHEX enzymatic activity on said substrate, wherein a difference in the amount of said product detected in the presence of said candidate compound as compared to that in the absence thereof is an indication that said candidate compound modulates PHEX. In a specific embodiment, the modulator is an inhibitor, and a lower amount of said product detected in the presence of said candidate compound as compared to that in the absence thereof is an indication that said candidate compound inhibits PHEX.

In further specific embodiments of these substrates and of methods of using these substrates, the amino acid residue at position P₁′ is aspartic acid. In further specific embodiments, the amino acid residue at position P₂′ is selected from the group consisting of a hydrophobic, an acidic and a polar amino acid residues. In further specific embodiments, the amino acid residue at position P₂′ is selected from the group consisting of an asparagine, a glutamine, a methionine, an alanine, a valine, a tryptophan, a threonine, a serine, a tyrosine, a phenylalanine, and an isoleucine residues. In further specific embodiments, the amino acid residue-at position P₂′ is selected from the group consisting of a tryptophan, a threonine, a serine, a tyrosine, a phenylalanine and an isoleucine residues. In further specific embodiments, the amino acid residue at position P₂ is not an arginine, a lysine, an asparagine or a glutamine residue. In further specific embodiments, P₂-P₁-P₁′-P₂′ is as set forth in SEQ ID NO:2 (phenylalanine-serine-aspartate-tyrosine). In further specific embodiments, the fluorophore unit is attached to P₃and the quencher unit is attached to P₃′. In further specific embodiments, the fluorophore unit is Abz, the quencher unit is Dnp, and P₃′ is a lysine residue. In further specific embodiments, the fluorophore unit is Abz and the quencher unit is Dnp, and Dnp is attached to a lysine residue. In further specific embodiments, the PHEX substrate has the chemical structure Abz-(SEQ ID NO:3)-Dnp.

The methods according to the present invention therefore include but are not limited to the use of a fluorogenic substrate. Indeed, it will be understood by a person of ordinary sktill in the art that cleavage of the peptide sequence units by PHEX according to the present invention can be detected by any means known in the art, in the methods of the present invention. These methods for detecting protease activity include for instance phase separation, column chromatography, HPLC as well a neo-epitope recognition and separation and Fluorescence Resonant Energy Transfer (FRET).

According to the specific embodiments of the present invention when fluorogenic substrates are used, the fluorophore unit can be any fluorogenic group (anthracene, aminobenzoyl, indole, aminoethylnaphthyl, and the like) which can be modified such as Abz, dansyl (5-dimethylamino-naphthalene-1-sulfonyl), nicotinic acid, 4-guanidino-benzoic acid, and derivatives thereof, e.g. N-methyl-Abz, 4-chloro-Abz, 5-chloro-Abz, 6-chloro-Abz, 3,5-dibromo-Abz; derivatives of nicotinic acid such as 6-amino-, 2-amino-, 2-chloronicotinic acid, and niflumic acid; derivatives of 4-guanidino-benzoic acid; derivatives of dansyl; and the like derivatives.

Similarly, the quencher unit can be any amino acid derivative which. has a quenching aromatic group which absorbs the fluorescence energy of the fluorophore unit and reduces the fluorescence emission when these units are covalently held in close proximity. Examples are Dnp, Trp, Tyr, Phe(p--NO2), Phe(m-NO2), and halogenated derivatives thereof. Therefore, although ortho-aminobenzoic acid (Abz) and 2,4-dinitrophenyl (Dnp) were used as the donor-acceptor pair Abz and Dnp as examples of fluorophore and quencher units of the present invention, other fluorophore and quencher units can be used to provide useful results in the fluorometric assays of the present invention.

Besides variations in the fluorophore and quencher units, the amino acids in the substrate sequences can be varied to optimize the affinity and kinetic properties for the particular PHEX under consideration. Alternatively, the positions of the fluorophore and quencher units can be interchanged in their position relative to the peptide sequence unit.

The peptide unit of the substrate preferably comprises at least 6 amino acid residues and can comprise as many as 15 amino acid residues. As the number of amino acids increases beyond that length, it appears that the increased distance between the fluorophore and quencher units tends to progressively decrease the subtrate's (( self-quenching )) ability. The peptide comprises either a glutamic or aspartic acid in the P₁′ position (according to the nomenclature of Schechter and Berger (37), and any hydrophobic, acidic or polar amino acid, preferably asparagine, glutamine, methionine, alanine, valine, threonine, serine, tryptophan, tyrosine, phenylalanine or isoleucine, and more preferably threonine, serine, tryptophan, tyrosine, phenylalanine or isoleucine, at the P₂′ position. A person skilled in the art will locate the P₁′, at least 2 amino acids distal to both the fluorophore and the quencher units and will select the first and the last amino acid of the peptide unit such that appropriate fluorophore and quencher units can be attached to said amino acid without hindering peptide synthesis. Furthermore, specific residues in P₂′ (Leu, Pro and Gly) and in Pi (lie, Val and His) positions preclude hydrolysis by secPHEX. Peptide Abz-GFSDYK(Dnp)-OH was nevertheless determined to contain the most favorable residues in the P₂ to P₂′ positions for use as a substrate for PHEX.

As used herein, the following abbreviations are used for the following terms: Abz, ortho-aminobenzoic acid; ADHR, autosomal dominant hypophosphatemic rickets; AFU, arbitrary fluorescence units, Dnp (2,4-dinitrophenyl); EDDnp, (N-[2,4-dinitrophenyl] ethylenediamine); FGF-23, fibroblast growth factor-23; Fmoc, N^{α}-fluoren-9-ylmethyloxycarbonyl; FRP-4, Frizzled-related protein-4; IQFPs, intemally quenched fluorogenic peptides; LLC-PK1 cells, porcine kidney cells; MALDI-TOF™, matrix-assisted-laser-desorption desorption ionization-time-of-flight; MEPE, matrix extracellular phosphoglycoprotein; MMEL2, membrane metallo-endopeptidase-like 2; PTHrP, parathyroid hormone-related peptide (PTHrP₁₀₇₋₁₃₉ means residues 107-139 of PTHrP);. TIO, tumor-induced osteomalacia; G: glycine; F: phenylalanine; R: arginine; D: aspartic acid; W: tryptophan; K: lysine; H: histidine; L: leucine; S: serine; T: threonine; Q: glutamine; Y: tyrosine; BSA: bovine serum albumin.

As used herein, the term “sample” refers to any biological sample. where it would be desirable to detect PHEX's presence or concentration. Without limiting the generality of the foregoing,. it includes biological medias such as serum, plasma, cell growth media, cell culture media, and media obtained from intermediary purification steps as well as for quality control.

As used herein the term “amino acid residue” includes not only natural but also unnatural amino acid residues. Substitution of unnatural amino acids for natural amino acids in the peptide sequences of the present invention can advantageously confer them more stability against degradation by exopeptidases contained in certain samples used in the methods of the present invention or any other type of degradation. Such a substitution can, for instance, confer resistance to proteolysis by exopeptidases acting on the N-terminus. Such substitutions have been described (Coller, et al. (1993), J. Biol. Chem., 26 8:20741-20743, incorporated herein by reference) and these substitutions do not affect biological activity so that PHEX will be able to cleave a peptide of the present invention constituted in whole or in part of unnatural amino acid residues. Furthermore, the synthesis of peptides with unnatural amino acids is routine and known in the art (see, for example, Coller, et al. (1993), supra).

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 graphically illustrates results of scanning for amino acid residues preferred by secPHEX in substrate positions P2 to P2′. Positional scanning fluorimetric combinatorial libraries with general sequences Abz-GXXZXK(Dnp)-OH (SEQ ID NO:4) (Panel A), Abz-GXXDZK(Dnp)-OH (SEQ ID NO:5) (Panel B), Abz-G)7DXK(Dnp)-OH (SEQ ID NO:6) (Panel C) and Abz-GZ)(DXK(Dnp)-OH (SEQ ID NO:7) (Panel D) were incubated with secPHEX (0.2 to 0.45 μM) in 0.1 M HEPES, 150 mM NaCl, pH 6.5, at 37° C. The assays were performed in low substrate concentration where the reactions followed first-order conditions ([S]<<Kₘ). The y axis represents the apparent catalytic efficiency values (*k_{cat}/Kₘ) obtained as described under the heading “Experimental Procedures” below. The x axis provides the specially addressed amino acid represented by the one letter code. The errors were less than 5% for any obtained value;

FIG. 2 graphically illustrates pH and NaCl (inset) dependence of Abz-GFSDYK(Dnp)-OH (SEQ ID NO:3) hydrolysis by secPHEX. * k,t/Kₘ values for the hydrolysis of Abz-GFSDYK(Dnp)-OH (SEQ ID NO:3) by PHEX were determined in presence of different buffers and pH conditions as described herein under the heading “Experimental Procedures” below. In the inset, the effect of NaCl is shown: Abz-GFSDYK(Dnp)-OH (SEQ ID NO:3) was incubated with secPHEX in 0.01 M Bis-Tris buffer, pH 5.5 with NaCl (0 to 0.5 M);

FIG. 3 graphically illustrates hydrolysis of Abz-GFSDYK(Dnp)-OH (SEQ ID NO:3) by secPHEX and membrane-bound PHEX. Abz-GFSDYK(Dnp)-OH (SEQ ID NO:3) at a concentration of 10 μM was incubated at 37° C., in 0.01 M Bis-Tris buffer, pH 5.5 with 150 mM NaCl, containing 2,1 μg total proteins from membranes extracted from LLC-PK1 transfected with vector alone (notes as “A” on the figure), or 2,1 μg total proteins from membranes extracted from LLC-PK1 transfected with the membrane-bound form of human PHEX (noted as “□” on the figure); or 2,1 μg total proteins from membranes extracted from LLC-PK1 transfected with vector alone to which 500 ng purified secPHEX were added (noted as “o” on the figure). Fluorescence was monitored every 4 min for a period of 28 min. Amounts of secPHEX or membrane-bound PHEX (mPHEX) in the enzymatic reactions were evaluated by immunoblotting (Inset). Mock: cells transfected with vector alone;

FIG. 4 graphically illustrates the activity of PHEX in the presence of NEP-specific substrate and inhibitor. NEP, secPHEX and membrane-bound PHEX (mPHEX) activities were measured after 30 minutes of incubation in presence of either 10 μM Abz-DRRL-EDDnp (SEQ ID NO:8) (A) or 10 μM Abz-GFSDYK(Dnp)-OH (SEQ ID NO:3) (B). Fluorescence detection (AFU) at γₑₘ=420 nm and γₑₓ=320 nm was determined as described under the heading “Experimental Procedures” below. When present, thiorphan, a NEP-specific inhibitor, was at a concentration of 10⁻⁶ M. Data are a compilation of three different experiments;

FIG. 5 schematically illustrates the positions of the synthetic peptides used as candidate substrates in MEPE and FGF-23structures. Panel A shows the MEPE structure: numbers correspond to the first and last amino acid residues of MEPE including the signal sequence. Dark box represents MEPE sequence homologous to sequences found in dentin matrix protein 1 (DMP1), dentin sialophosphoprotein (DSPP) and osteopontin (OPN). Boxes under MEPE structure correspond to the position in MEPE sequence of the peptides presented in Table IV. Numbering is as provided in Table IV. Panel B shows the FGF-23structure: numbers correspond to the first and last amino acid residues of FGF-23including the signal sequence. RHTR (SEQ ID NO:9)indicate the position of the convertase cleavage site found in FGF-23sequence. Boxes under FGF-23structure correspond to the position in FGF-23sequence of the peptides presented in Table lIl. Numbering is as in Table IlIl;

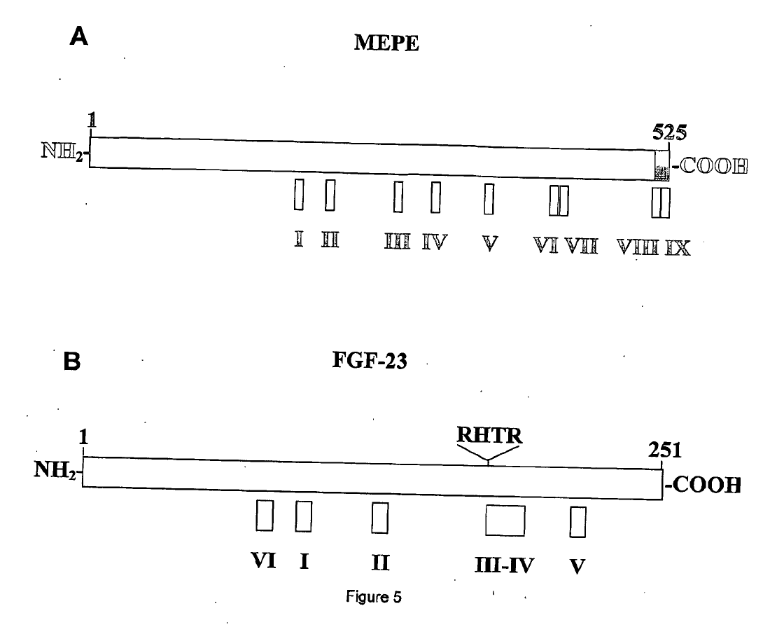

FIG. 6 graphically illustrates the secPHEX enzymatic activity on a fluorigenic substrate of the present invention in rat serum. The observed fluorescence increase was proportional to the reaction product formation. The reaction was carried out in a total volume of 200 μl constituted of buffer: 50 mM Hepes (NaOH) pH 7.4, 150 mM NaCl, 5*10−6 M captopril (ACE inhibitor), containing 10 μl of rat serum in which purified sPHEX was spiked. The enzymatic reaction was initiated by the addition of the fluorigenic substrate (Abz-GFSDYK-Dnp) (SEQ ID NO:3) at a final concentration of 20 μM. The fluorescent reaction product was excited at 320 nm and fluorescence recorded at 420 nm every four minutes over a one-hour period in a 96-well plate reader (Perkin-Elmer, HTS-7000). The initial enzymatic speed rate of the reaction was calculated for each condition. The reaction was carried out in presence of 5 mM EDTA to assess whether the assay is specific to metallopeptidase. Measurements were done in duplicates.

### DESCRIPTION OF THE SPECIFIC EMBODIMENTS

Combinatorial fluorescent-quenched peptide libraries constituted of a sequence of 3 units that comprise a fluorophore unit, a peptide unit and a quencher unit were synthesized, and their members tested as PHEX substrates.

### EXPERIMENTAL PROCEDURES

### Expression and Purification of Recombinant PHEX and NEP

The human recombinant secPHEX was obtained following the procedure previously described (17, WO 00/50580). Purified secPHEX concentration was determined using the Bradford method (DC protein assay kit; Bio-Rad, Mississauga, ON, Canada). Membrane-bound recombinant human PHEX was expressed in LLC-PK1 cells (17, (WO 00/50580)) and membranes were prepared essentially as described by Guo et al. (34). Briefly, transfected cells resuspended in lysis buffer (150 mM NaCl, 20 mM Tris.HCl, pH 7.6) were disrupted by sonication. Cell debris were removed by centrifugation at 300 g for 15 min at 4 °C. Membranes were collected by centrifugation at 30,000 g for 50 min and solubilized for 2h with 1% n-dodecyl-β-D-maltoside in lysis buffer. Insoluble material was pelleted by centrifugation at 10,000 g for 15 min and protein concentrations of the supernatant determined using the Bradford method described above. PHEX amounts were evaluated by immunoblotting as described previously (17). NEP expression and purification were performed as described previously (36).

### N-terminal Sequence Determination

Purified secPHEX was analyzed by SDS-PAGE on a 10.0% gel (37). Proteins were transferred to a polyvinylidine difluoride microporous (PVDF) membrane (Immobilon O^{TM}, Millipore), stained with Coomassie Blue, destained and washed extensively with distilled water. The bands were excised and the N-terminal sequence determined in a PPSQ-23protein sequencer (Shimadzu Tokyo, Japan).

### Synthesis of Peptide Libraries

Positional scanning IQFP combinatorial libraries were synthesized by the methods previously described (38, 39), with the exception that Abz/Dnp was used as fluorescence donor/acceptor pair. For preliminary experiments, a library with the general structure Abz-GXXZXK(Dnp)-OH (SEQ ID NO:4) was prepared, where the Z position was successively filled with any of the 20 naturally occurring amino acids (except cysteine to avoid dimerization) and X contained randomly incorporated these 19 residues. To ensure equal coupling of the randomized residues, a balanced mixture of 19 amino acids was used following the optimum composition previously described (40). Three other libraries were prepared with structures Abz-GXXDZK(Dnp)-OH (SEQ ID NO:5), Abz-GXZDXK(Dnp)-OH (SEQ ID NO:6) and Abz-GZXDXK(Dnp)-OH (SEQ ID NO:7), in which the P₁′ position was pre-fixed as Asp. Z, in positions P₂′, P₁ and P₂, respectively in these libraries was any of the 20 naturally occurring amino acids (except cysteine to avoid dimerization). The other positions “X” were randomized (X=17 amino acids; Cys, Asp and Glu were excluded of the mixture in order to force the hydrolysis only at the fixed residue). Stock solutions were prepared in DMSO and the concentrations were measured using the Dnp molar extinction coefficient ε₃₆₅=17,300 M⁻¹ cm⁻¹.

Synthesis and Purification of Peptides

IQFPs containing the group EDDnp attached to a glutamine residue, were synthesized by the solid-phase synthesis method as previously described (41) in a Shimadzu model PSSM-8 automated solid-phase peptide synthesizer. The IQFPs containing the Dnp group incorporated to the ε-NH2 of a Lys residue, were synthesized by the solid-phase methodology, using Fmoc-Lys(Dnp)-OH to introduce the quencher group. All the peptides obtained were purified by semi-preparative HPLC. The molecular weight and purity of synthesized peptides were checked by amino acid analysis and by molecular weight determination with MALDI-TOF™ mass spectrometry, using a TofSpec E™ from Micromass, Manchester, U.K. Stock solutions of Dnp- or EDDnp-peptides were prepared in DMSO and the concentrations were measured using the Dnp molar extinction coefficient ε₃₆₅=17,300 M⁻¹ cm⁻¹, as above.

Peptide Library Screen

Hydrolysis of peptides in the library was monitored under optimum pH and salt concentrations previously established for recombinant secPHEX using PTHrP₁₀₇₋₁₃₉ as substrate (17), namely 0.1 M HEPES, pH 6.5, containing 0.15 M NaCl. Enzymatic activity in a final volume of 0.35 ml was continuously followed at 37° C. in a Hitachi F-2000 fluorimeter by measuring the fluorescence at γₑₘ=420 nm and γₑₓ=320 nm. The assays were performed at low substrate concentrations where the reactions followed first-order conditions ([S]<<Kₘ), and the rate constant (k_{obs}) was determined by the non-linear regression data analysis Grafit program (42). These determinations were done at two different substrate concentrations and the apparent catalytic efficiencies, designated *k_{cat}/Kₘ, were obtained by dividing the k_{obs} by the enzyme concentration. The error was less than 5% for any obtained value.

Optimum pH Determination

The pH dependence was studied using 0.2 pM of Abz-GFSDYK(Dnp)-OH (SEQ ID NO:3) over a pH range of 3.5 to 8.5. The buffers used were as follows: 0.01 M sodium acetate (3.5<pH<5.1), 0.01 M Bis-Tris (5.1<pH<6.5), 0.01 M Hepes (6.5<pH<7.4) and 0.01 M Tris-HCI (7.4<pH<8.4), containing 0.14 M NaCl. Enzymatic activity was followed at 37° C. using the continuous fluorimetric assay and the apparent second-order rate constant (*k_{cat}/Kₘ) was calculated as described above for each pH.

NaCl Influence on Catalytic Activity

The influence of salt on secPHEX catalytic activity was investigated using 0.2 μM of Abz-GFSDYK(Dnp)-OH (SEQ ID NO:3) as substrate at 37° C., in 0.01 M Bis-Tris buffer, pH 5.5, over a NaCl range of 0 to 500 mM. The increase in fluorescence was continuously measured at 37° C. and the apparent second-order rate constant (*k_{cat}/Kₘ) for the different NaCl concentrations was calculated as described above.

Determination of Kinetic Parameters for Synthetic Peptides from the Peptide Libraries and from Putative Natural Substrates

The hydrolysis of the IQFPs, at 37° C., in 10 mM Bit-Tris buffer pH 5.5 (0.35 to 1.0 ml final volume) containing 150 mM NaCl, was continuously followed measuring the fluorescence at γₑₘ=420 nm and γₑₓ=320 nm in a Hitachi F-2000™ spectrofluorometer. The cuvefte containing the buffer and the substrate was placed in a thermostatically controlled cell compartment for 5 min before the addition of the enzyme and the increase in fluorescence with time was continuously recorded for 5-10 min. The enzyme concentration for initial rate determination was chosen at a level intended to hydrolyze less than 5% of the substrate present. The slope was converted into micromoles of substrate hydrolyzed per min based on a calibration curve obtained from complete hydrolysis of each peptide. The inner-filter effect was corrected using an empirical equation as previously described (43). The kinetic parameters Kₘ and k_{cat} were calculated by the non-linear regression data analysis Grafit program (42). The k_{cat}/Kₘ values were calculated as the ratio of these two determined parameters. The apparent second order rate constant k_{cat}/Kₘ (*kIt/Kₘ) was determined under pseudo first-order conditions, where [S]<<Kₘ. These determinations were performed in two different substrate concentrations and the errors were less than 5% for any obtained value.

Determination of secPHEX Cleavage Sites

To determine the position of cleavage in peptides of the IQFPs combinatorial libraries, the products resulting from hydrolysis by secPHEX were submitted to N-terminal amino acid sequencing in a PPSQ-23protein sequencer (Shimadzu Tokyo, Japan). The scissile bonds in the IQFPs secPHEX substrates derived from PTHrP₁₀₇₋₁₃₉, FGF-23and MEPE were determined by amino acid sequencing and by MALDI-TOF™ mass spectrometry (TofSpec-E™, Micromass, Manchester, U.K.) after isolation of the fragments resulting from the hydrolysis by secPHEX by analytical HPLC.

### EXAMPLE 1

### secPHEX Specificity Determined by Peptide Libraries

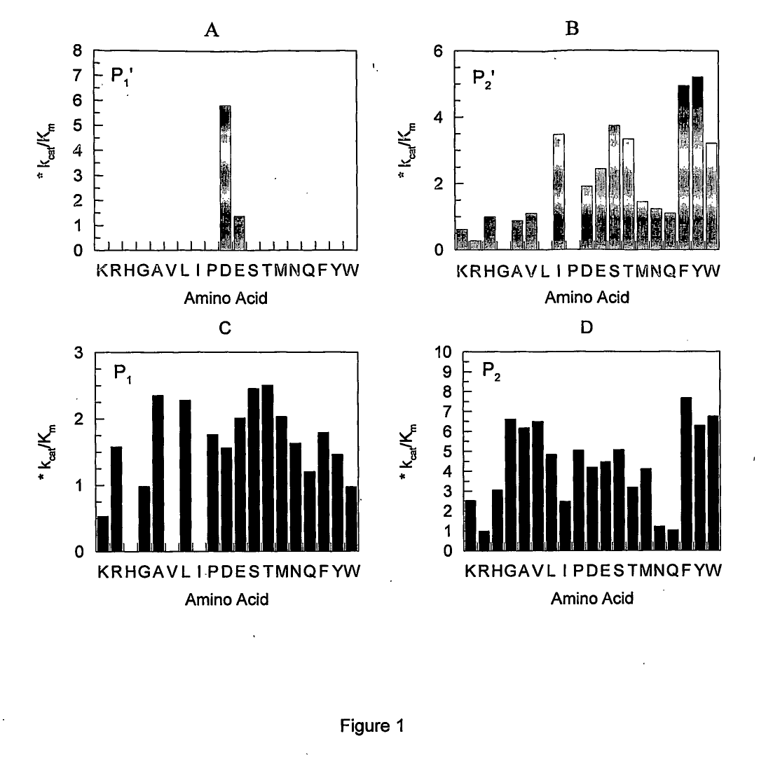

Positional scanning synthetic combinatorial libraries were used to identify the P₂ to P₂′ substrate specificity of secPHEX. P₂ to P₂′ are defined according to the nomenclature of Schechter and Berger (44). secPHEX was determined to require an acidic residue in P₁′ position as demonstrated using the library with the general sequence Abz-GXXZXK(Dnp) (SEQ ID NO:4), where only the peptides Abz-GXXDXK(Dnp) (SEQ ID NO:10) and Abz-GXXEXK(Dnp) (SEQ ID NO:11) were hydrolyzed (FIG. 1, panel A). The products resulting from cleavage were submitted to N-terminal amino acid sequencing and the presence of an aspartate or a glutamate as the first residue of the fragments DXK(Dnp) and EXK(Dnp), respectively, was confirmed. However, a clear preference was observed for an Asp in this. position as substitution by a Glu resulted in almost six-fold reduction on the catalytic efficiency (*k_{cat}/Kₘ) (FIG. 1, panel A).

Based on the results from the first library, three other libraries with general structures Abz-GXXDZK(Dnp) (SEQ ID NO:5), Abz-GXZDXK(Dnp) (SEQ ID NO:6) and Abz-GZXDXK(Dnp) (SEQ ID NO:7), in which P₁′ was fixed as Asp, were synthesized and tested with recombinant secPHEX to determine the S₂′, S₁ and S₂ specificities, respectively. The P₂′ position showed a moderate preference for certain amino acids including those with aromatic side chains such as Phe and Tyr as well as for polar residues such as Ser and Thr (FIG. 1, panel B). In contrast, substrates containing Leu, Pro and Gly in this position were resistant to hydrolysis by secPHEX. The S₁ subsite accepted a broad range of amino acids although substrates containing IIe, Val and His in this position were resistant to hydrolysis (FIG. 1, panel C). No impeditive residues were detected in P₂ position since secPHEX was able to hydrolyze all substrates of the Abz-GZXDXK(Dnp) (SEQ ID NO:7) series. However, in this library, peptides containing the amino acids Arg, Lys, Asn and Gin in P₂ were the least preferred substrates (FIG. 1, panel D).

A model peptide containing the most favorable amino acid residue in each position screened by the libraries was next synthesized. The resulting sequence, Abz-GFSDYK(Dnp)-OH (SEQ ID NO:3), was used to better characterize the enzyme and to establish the optimum assay conditions for the kinetic studies.

### EXAMPLE 2

### pH Activity Profiles and NaCl Dependence

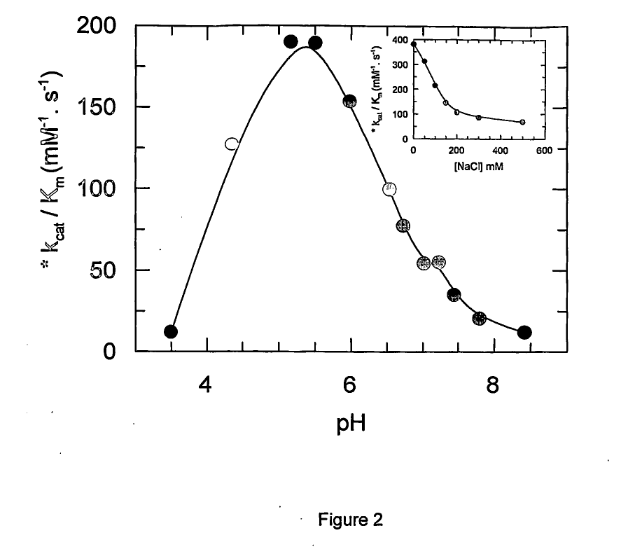

The effect of pH on the hydrolysis of Abz-GFSDYK(Dnp)-OH (SEQ ID NO:3) by secPHEX was determined over a pH range of 3.5 to 8.5. A bell shaped curve was obtained with maximum k_{cat}/Kₘ values occurring around pH 5.5 (FIG. 2). A significant influence of NaCl concentration on the catalytic efficiency of recombinant secPHEX was also detected, as shown in the inset of FIG. 2. kKt/K values in presence of 10 mM Bis Tris, pH 5,5 containing 150 mM NaCl were more than 2-fold lower than in the absence of added salt. However, the presence of the salt is desirable for the Kₘ and k_{cat} determinations when the assays is not performed immediately or in a relatively short period of time since in absence of NaCl the enzyme is gradually inactivated (data not shown).

### EXAMPLE 3

Effects of substrate COOH-terminus modification

The effect of amidating the COOH-terminus of substrates on cleavage efficiency by secPHEX was then considered. Two substrates, Abz-GFSDYK(Dnp)-OH (SEQ ID NO:3) and Abz-GFSEYI<(Dnp)-QH (SEQ IDNO:12) (peptides I and IV, Table I), and their COOH-terminus amidated analogues (peptides 11 and V, Table I) were used to compare the activity of secPHEX. The kinetic parameters presented in Table I show that the model peptide Abz-GFSDYK(Dnp)-OH (SEQ ID NO:3) (peptide 1, Table I) was the best substrate of the series (kat/Kₘ=166.7 mM⁻¹.s⁻¹) due to its high affinity for PHEX. On the other hand, its amidated analogue Abz-GFSDYK(Dnp)-NH2 (SEQ ID NO:3) (peptide 11, Table I) was hydrolyzed by the enzyme with a high k_{cat} value but with a 6-fold decrease in catalytic efficiency due to its low affinity for the enzyme. As expected from the results described above, secPHEX catalytic efficiency was lower with Abz-GFSEYI<(Dnp)-OH (SEQ ID NO:12) (peptide IV, Table I), containing Glu in P₁′ and a free carboxyl group, than that observed with the Asp-containing analogue (peptide 1, Table I). Abz-GFSEYK(Dnp)-NH2 (SEQ ID NO:12) (peptide V, Table I) was resistant to hydrolysis confirming that substrates with blocked COOH-terminal groups are less susceptible to hydrolysis by secPHEX. Replacing K(Dnp) by Q-EDDnp at the COOH-terminal end of the model peptide resulted in a decrease of the k_{cat}/Kₘ value for peptide Abz-GFSDYQ-EDDnp (SEQ ID NO:13) (compare peptides I and ll, Table I). Q-EDDnp is present in peptides prepared by the solid-phase synthesis method.

TABLE I

KINETIC CONSTANTS FOR THE HYDROLYSIS BY PHEX
OF FLUOROGENIC PEPTIDES DESIGNED BY SCREENING
COMBINATORIAL LIBRARIES

| | Kₘ | k_{cat} | k_{cat}/Kₘ |
|---|---|---|---|
| Peptide | (μM) | (s⁻¹) | (mM⁻¹ s⁻¹) |

| | | | | |
|---|---|---|---|---|
| I-Abz-GFS↓DYK(Dnp)-OH | (SEQ ID NO:3) | 3.0 | 0.5 | 166.7 |
| II-Abz-GFS↓DYK(Dnp)-NH₂ | (SEQ ID NO:3) | 53.2 | 1.3 | 24.4 |
| III-Abz-GFS↓DYQ-EDDnp | (SEQ ID NO:13) | 13.3 | 0.6 | 45.1 |
| IV-Abz-GFS4↓EYK(Dnp)-OH | (SEQ ID NO:12) | | | 9.0* |

| | | |
|---|---|---|
| V-Abz-GFSEYK(Dnp)-NH₂ | (SEQ ID NO:12) | Resistant |

*k_{cat}/Kₘ value determined under pseudo first-order conditions.

The assays were performed at 37° C., in 10 mM Bis-Tris buffer pH 5.5, containing 150 mM NaCl. Measurements were made as described under the heading “Experimental Procedures” above. Cleavage sites are indicated as arrows (↓). The standard deviations of the kinetic constants were less than 5%.

### EXAMPLE 4

### Substrates Containing Sequences Based on PTHrP₁₀₇₋₁₃₉

SecPHEX was reported to cleave PTHrP₁₀₇₋₁₃₉ at three positions (17). To determine cleavage efficiency of this substrate, peptides with sequences encompassing the identified hydrolyzed peptide bonds were synthesized and incubated with secPHEX. The enzyme had a high affinity for these peptides (low Kₘ values) but low Scat values (Table II). Despite the low catalytic constant, peptide Abz-D₁₂₄-HLSDTSTQ-EDDnp (SEQ ID NO:14) (peptide I, Table II) was hydrolyzed with k_{cat}/Kₘ value of 77.0 mM⁻¹.s⁻¹, being among the good substrates for secPHEX described herein. In all cases, hydrolysis of the substrates occurred in amino-terminus of an Asp residue.

TABLE II

KINETIC CONSTANTS FOR HYDROLYSIS BY PHEX
OF IQFPs BASED ON PTHrP₁₀₇₋₁₃₉ SEQUENCE

| | Kₘ | k_{cat} | k_{cat}/Kₘ |
|---|---|---|---|
| Peptide | (μM) | (s⁻¹) | (mM⁻¹ s⁻¹) |

| | | | | |
|---|---|---|---|---|
| I-Abz-D₁₂₅-HLS↓DTSTQ-EDDnp^{a} | (SEQ ID NO:14) | 1.3 | 0.1 | 77.0 |
| II-Abz-L₁₃₄-EL↓-DSRQ-EDDnp | (SEQ ID NO:15) | 0.9 | 0.05 | 55.5 |
| III-Abz-A₁₁₀-WL↓-DSGVQ-EDDnp | (SEQ ID NO:16) | 1.5 | 0.04 | 26.7 |

^{a}The numbers in the peptide sequences identify the position of the residues in PTHrP₁₀₇₋₁₃₉

The assays were performed at 37° C., in 10 mM Bis-Tris buffer pH 5.5, containing 150 mM NaCl. Measurements were made as described under the heading “Experimental Procedures” above. Cleavage sites are indicated by arrows (↓). The standard deviations of the kinetic constants were less than 5%.

### EXAMPLE 5

Hvdrolysis of Peptides based on Human FGF-23Sequence

FGF-23has been proposed as a PHEX substrate (45). Using the information gathered from the combinatorial libraries about the most favorable amino acids in P₂ to P₂′ positions, the FGF-23sequence (31) was scanned for putative cleavage sites and IQFPs analogues were synthesized. Residues of Cys found in human FGF-23sequence were substituted by Met to avoid synthesis problems. In spite of the presence of more than one putative scissile bond in some peptides, all substrates of this series were hydrolyzed at a single cleavage site at the N-terminus of Asp residues, as determined by amino-terminal sequencing. The kinetic parameters presented in Table IlI reveal that Abz-R₁₇₅-RHTRSAEDDSERQ-EDDnp (SEQ ID NO:17) (peptide Ill, Table ll), the longest substrate tested showed the highest k_{cat}/Kₘ value of this series due to its high affinity for the enzyme. The peptide Abz-L₉₄-MMDFRGQ-EDDnp (SEQ ID NO:18) (peptide I, Table III) had a low affinity for the enzyme but the highest k_{cat}value. Peptides Abz-S₂₁₂-AEDNSPQ-EDDnp (SEQ ID NO:19) and Abz-R₇₆-SEDAGFQ-EDDnp (SEQ ID NO:20) (peptides V and VI, Table III) presented poor catalytic efficiencies and Abz-N₁₂₂-GYDVYHQ-EDDnp (SEQ ID NO:21) (peptide II, Table II) was the poorest substrate of this series.

TABLE III

KINETIC CONSTANTS FOR HYDROLYSIS BY PHEX OF INTERNALLY
QUENCHED FLUOROGENIC PEPTIDES BASED ON FGF23 SEQUENCE

| | Kₘ | k_{cat} | k_{cat}/Kₘ |
|---|---|---|---|
| Peptide | (μM) | (s⁻¹) | (mM⁻¹ s⁻¹) |

| | | | | |
|---|---|---|---|---|
| I-Abz-L₉₄-MM↓DFRGQ-EDDnp^{a} | (SEQ ID NO:18) | 17.0 | 0.8 | 45.0 |
| II-Abz-N₁₂₂-GY↓DVYHQ-EDDnp | (SEQ ID NO:21) | 9.0 | 0.01 | 1.1 |
| III-Abz-R₁₇₅-RHTRSAED↓DSERQ-EDDnp | (SEQ ID NO:17) | 3.0 | 0.2 | 66.7 |
| IV-Abz-R₁₇₅-RHTQSAED↓DSERQ-EDDnp | (SEQ ID NO:22) | 1.7 | 0.15 | 88.2 |
| V-Abz-S₂₁₂-AE↓DNSPQ-EDDnp | (SEQ ID NO:19) | 22.0 | 0.3 | 13.6 |
| VI-Abz-R₇₆-SE↓DAGFQ-EDDnp | (SEQ ID NO:20) | 4.1 | 0.06 | 14.6 |

^{a}The numbers in the peptide sequences identify the position of the residues in FGF23 protein.

The assays were. performed at 37° C., in 10 mM Bis-Tris buffer pH 5.5, containing 150 mM NaCl. Measurements were made as described under the heading “Experimental Procedures” above. Cleavage sites are indicated by arrows (↓). The standard deviations of the kinetic constants were less than 5%.

### EXAMPLE 6

### Hydrolysis of Peptides Based on Human MEPE Sequence

As was done for FGF-23, the human MEPE sequence (33) was mapped and IQFPs derivatives containing Asp or Glu were synthesized and tested as secPHEX substrates. Again, all peptides were hydrolyzed at a single cleavage site in the amino-terminus of an Asp residue as presented in Table IV. The majority of the substrates of this series were hydrolyzed with poor catalytic efficiency due to low k_{cat} and high Kₘ values. However, peptides Abz-G₃₈₆-SSDAAEQ-EDDnp (SEQ ID NO:23) and Abz-R₅₀₆-RDDSSEQ-EDDnp (SEQ ID NO:24) (peptides V and VIII, Table IV), showed the best k_{cat}/Kₘ values due mainly to their high affinity for the enzyme. Interestingly, peptide Abz-I₂₃₂-PSDFEGQ-EDDnp (SEQ ID NO:25) (peptide 11, Table IV) containing Ser in P₁ and Phe in P₂′, found to be well accepted residues in these positions by the library studies, exhibited the highest Kₘ value of the series, but the highest catalytic constant among all the peptides for which results are presented herein.

TABLE IV

KINETIC CONSTANTS FOR HYDROLYSIS BY PHEX OF INTERNALLY
QUENCHED FLUOROGENIC PEPTIDES BASED ON MEPE SEQUENCE

| | Kₘ | k_{cat} | k_{cat}/Kₘ |
|---|---|---|---|
| Peptide | (μM) | (s⁻¹) | (mM⁻¹ s⁻¹) |

| | | | | |
|---|---|---|---|---|
| I-Abz-GP₁₉₇-QR↓DSQAQ-EDDnpa | (SEQ ID NO:26) | 47.0 | 0.2 | 4.3 |
| II-Abz-I₂₃₂-PS↓DFEGQ-EDDnp | (SEQ ID NO:25) | N/D | N/D | 80.0* |
| III-Abz-T₂₉₄-HL↓DTKKQ-EDDnp | (SEQ ID NO:27) | 15.0 | 0.02 | 1.3 |
| IV-Abz-G₃₃₇-SN↓DIMGQ-EDDnp | (SEQ ID NO:28) | 13.0 | 0.01 | 0.8 |
| V-Abz-G₃₈₆₋SS↓DAAEQ-EDDnp | (SEQ ID NO:23) | 2.4 | 0.3 | 125.0 |
| VI-Abz-R₄₄₁-GL↓DNEIQ-EDDnp | (SEQ ID NO:29) | 7.0 | 0.03 | 4.3 |
| VII-Abz-N₄₄₉-EM↓DSFNQ-EDDnp | (SEQ ID NO:30) | 38.0 | 0.3 | 7.9 |
| VIII-Abz-R₅₀₆-RD↓DSSEQ-EDDnp | (SEQ ID NO:24) | 5.3 | 0.5 | 94.3 |
| IX-Abz-S₅₁₃-S↓DSGSQ-EDDnp | (SEQ ID NO:31) | 31.6 | 0.2 | 7.1 |

^{a}The numbers in the peptide sequences identify the residues as in MEPE.
*k_{cat}/Kₘ value determined under pseudo-first order conditions.

The assays were performed at 37° C., in 10 mM Bis-Tris buffer pH 5.5, containing 150 mM NaCl. Measurements were made as described under “Experimental Procedures”. Cleavage sites are indicated by arrows (↓). The standard deviations of the kinetic constants were less than 5%.

### EXAMPLE 7

### Comparison of Membrane-bound PHEX and secPHEX Activity

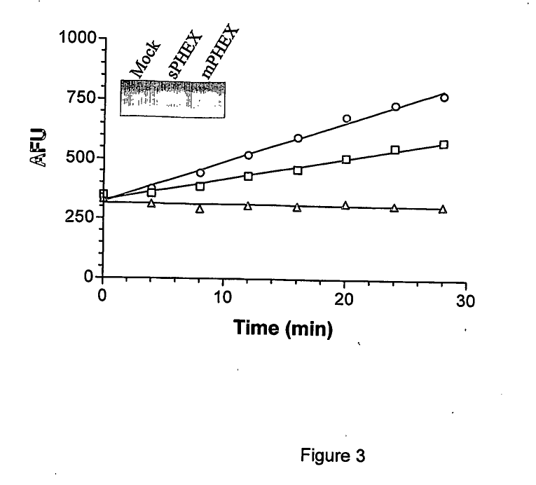

It can be argued that engineering a soluble form of PHEX may change the specificity of the enzyme. To rule out this possibility, LLC-PK1 cells were then transfected with membrane-bound PHEX cDNA and PHEX activity in the membrane fraction was compared to secPHEX, using Abz-GFSDYlID(np)-OH (SEQ ID NO:3) as substrate. To obtain a better comparison between secPHEX and membrane-bound PHEX in this series of experiments, secPHEX was added to membranes purified from cells transfected with vector alone. When similar amounts of secPHEX and membrane-bound PHEX, as evaluated by immunoblotting (FIG. 3, inset), were added to the enzymatic assay, degradation of the substrate was observed (FIG. 3). No substrate hydrolysis was evident with membranes from cells transfected with vector alone. A higher rate of hydrolysis was observed with secPHEX, which also showed a slightly lower Kₘ value (5×10⁻⁶ M and 10×10⁻⁶ M, for secPHEX and membrane-bound PHEX, respectively). Amino-terminal sequencing of the hydrolysis products showed that both enzymes cleaved substrate Abz-GFSDYK(Dnp)-OH (SEQ ID NO:3) at the amino-terminus of the Asp residue.

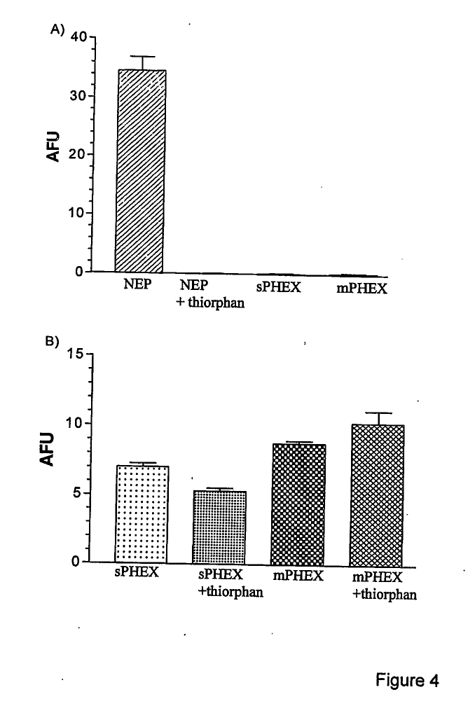

The activities of membrane-bound PHEX and secPHEX were also compared with the NEP-specific substrate Abz-DRRL-EDDnp (SEQ ID NO:8) (46). Neither enzyme hydrolyzed this substrate (FIG. 4, panel A). In addition, the NEP-specific inhibitor thiorphan did not prevent cleavage of substrate Abz-GFSDYK(Dnp)-OH (SEQ ID NO:3) by membrane-bound or secPHEX (FIG. 4, panel B). In contrast, NEP activity was completely inhibited by the same concentration of thiorphan (FIG. 4, panel A). These results demonstrated that engineering human PHEX into a soluble enzyme did not affect its activity and specificity, and that PHEX does not display a NEP-like activity.

Furthermore, these results support the applicants previous observation that PHEX could not cleave several well-known NEP substrates, including enkephalins and substance P (17). Thus, the present invention clearly shows that PHEX and NEP have different substrate specificities, and is not in agreement with reports that PHEX is able to hydrolyze NEP substrates, such as Leu-enkephalin (34) and the chromogenic peptide Z-AAL-pNA (35), as these peptides do not have acidic residues in their amino acid sequences.

### EXAMPLE 8

### Determination of secPHEX Activity and Concentration in Serum Using Substrate of the Present Invention

secPHEX was purified to homogeneity according to the methods of Crine and Boileau (WO 00/50580)). secPHEX quantity was measured with the Bradford assay (BioRad) using BSA as a standard.

secPHEX enzymatic activity was determined using a fluorigenic substrate where increase of fluorescence was proportional to substrate conversion to products. At least three fluorigenic substrates were shown to be good secPHEX substrate for this application: Abz-GFRDWK-Dnp (SEQ ID NO:32) (S1), Abz-DHLSDTSTQedDnp (SEQ ID NO:14) (S2) and Abz-GFSDYK-Dnp (SEQ ID NO:3) (S3). Of these, S3 was the most preferred substrate. S3 was synthesized at Dr. Gilles Lajoie laboratories (University of Western Ontario, Canada) using the technique of Fmoc solid-phase peptide synthesis with commercially available protected amino acids. This technique is well known to those versed in the art. (see for example: Atherton E. and Sheppard R. C. (1987) in The Peptides, Vol. 9, p. 1, editors, S. Udenfriend and J. Meienhofer, Academic Press, New York and, Fmoc Solid Phase Peptide Synthesis; A Practical Approach (2000), editors, W. C. Chan and P. D. White, Oxford University Press).

The reaction was carried out in a total volume of 200 μl constituted of buffer (50 mM Mes(NaOH) pH 6.5, 150 mM NaCl, 5×10⁻⁶ M captopril (ACE inhibitor)) and serum (preferably between 2 to 50 μl). The enzymatic reaction was initiated with the addition of the fluorigenic substrate at a final concentration of 10 μM. The fluorescent reaction product is excited at 320 nm with fluoresce emission read at 420 nm every four minutes over a one-hour period using 96-well plate reader (Perikin-Elmer, HTS-7000™). The initial reaction rate was calculated from the acquired fluorescence-time data as a slope using Microsoft Excell™. PHEX Enzymatic activity (Vi) was calculated by subtracting to the initial rate of fluorescence (Vitot) with the same measured in presence of EDTA 5 mM (ViEDTA).
Vi=Vitot−ViEDTA

The actual secPHEX concentration was determined from a calibration curve (Vi Vs secPHEX concentration) obtained by serial dilution of secPHEX in serum or other biological media as required.

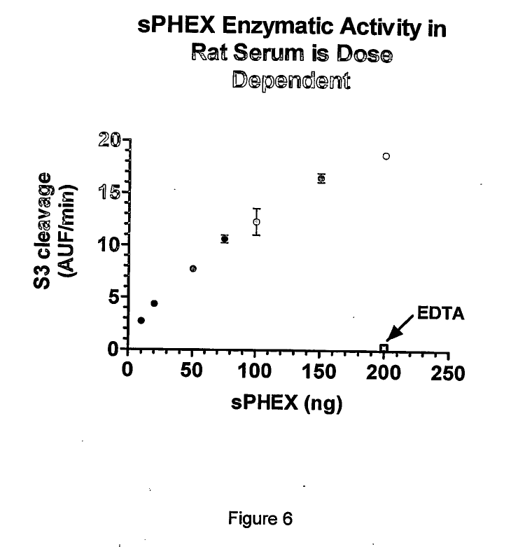

Results of this assay are presented in FIG. 6.

### EXAMPLE 9

### IC₅₀ Determination of PHEX Inhibitor Using Substrates of the Present Invention

secPHEX was purified to homogeneity according to the methods described above. secPHEX concentration was measured with the Bradford assay (BioRad) using BSA as a standard.

secPHEX enzymatic activity was determined using a fluorigenic substrate where increase of fluorescence was proportional to substrate conversion to products. At least three fluorigenic substrates were shown to be good secPHEX substrate for this application: Abz-GFRDWK-Dnp (SEQ ID NO:32) (S1), Abz-DHLSDTSTQ-edDnp (SEQ ID NO:14) (S2) and Abz-GFSDYK-Dnp (SEQ ID NO:3) (S3). Of these, S3 was the most preferred substrate. S3 was synthesized as described above.

The reaction was carried out in a total volume of 200 μl constituted of buffer (50 mM Mes(NaOH) pH 6.5, 150 mM NaCl) containing various concentrations of the tested PHEX inhibitor (3×10⁻⁵ to 1×10⁻¹¹ M) and 100 ng of purified secPHEX. The enzymatic reaction was initiated with the addition of the fluorigenic substrate at a final concentration of 10 μM. The fluorescent reaction product was excited at 320 nm with fluoresce emission read at 420 nm every four minutes over a one-hour period using 96-well plate reader (Perkin-Elmer, HTS-7000™). The initial reaction rate was calculated from the acquired fluorescence-time data as a slope using Microsoft Excell™. Further examples can be found in the co-pending PCT application number PCT/CA03/01893.

IC₅₀ was determined with a plot of the initial rates computed above as a function of inhibitor concentration using the one site competition equation of GraphPad Prism™ version 3.03 (GraphPad™ Software Inc.).

Although the present invention has been described hereinabove by way of preferred embodiments thereof, it can be modified, without departing from the spirit and nature of the subject invention as defined in the appended claims.

1. A fluorogenic PHEX substrate comprising
a peptide unit;
a fluorophore unit capable of conferring fluorescence on said substrate attached to an amino acid residue at a first end of the peptide unit; and
a quencher unit capable of providing intramolecular quenching of said fluorescence attached to an amino acid residue at a second end of the peptide unit;
the peptide unit having at least 6 amino acids residues including a sequence P₂-P₁-P₁′-P₂′ of 4 amino acid residues at positions P₂, P₁, P₁′ and P₂′ of the peptide unit, respectively; the amino acid residue at position P₂ being any amino acid residue; the amino acid residue at position P₁ being any amino acid residue except an isoleucine, a valine, or a histidine residue; the amino acid residue at position P₁′ being an acidic amino acid residue selected from the group consisting of a glutamic acid residue and an aspartic acid residue, and being located at least 2 amino acid residues distal to both the fluorophore and the quencher units; the amino acid residue at position P₂′ being any amino acid residue except a leucine, a proline or a glycine residue, with the proviso that said peptide unit does not have the sequence as set forth in SEQ ID NO: 1.

2. A fluorogenic PHEX substrate as recited in claim 1, wherein said amino acid residue at position P₁′ is aspartic acid.

3. A fluorogenic PHEX substrate as recited in claim 1, wherein said amino acid residue at position P₂′ is selected from the group consisting of a hydrophobic, an acidic and a polar amino acid residues.

4. A fluorogenic PHEX substrate as recited in claim 1, wherein said amino acid residue at position P₂′ is selected from the group consisting of an asparagine, a glutamine, a methionine, an alanine, a valine, a tryptophan, a threonine, a serine, a tyrosine, a phenylalanine, and an isoleucine residues. Customer No.: 24280

5. A fluorogenic PHEX substrate as recited in claim 1, wherein said amino acid residue at position P₂′ is selected from the group consisting of a tryptophan, a threonine, a serine, a tyrosine, a phenylalanine and an isoleucine residues.

6. A fluorogenic PHEX substrate as recited in claim 1, wherein said amino acid residue at position P₂ is not an arginine, a lysine, an asparagine or a glutamine residue.

7. A fluorogenic PHEX substrate as recited in claim 1, wherein said P₂-P₁-P₁′-P₂′ is as set forth in SEQ ID NO:2.

8. A fluorogenic PHEX substrate as recited in claim 1, wherein the fluorophore unit is Abz and the quencher unit is Dnp, and wherein Dnp is attached to a lysine residue.

9. A fluorogenic PHEX substrate having the chemical structure Abz-(SEQ ID NO:3)-Dnp.

10. A method for identifying a PHEX modulator comprising contacting a candidate compound with PHEX in the presence of a PHEX substrate, said substrate including a peptide unit of at least 6 amino acids residues including a sequence P₂-P₁-P₁′-P₂′ of 4 amino acid residues at positions P₂, P₁, P₁′ and P₂′ of the peptide unit, respectively; the amino acid residue at position P₂ being any amino acid residue; the amino acid residue at position P₁ being any amino acid residue except an isoleucine, a valine, or a histidine residue; the amino acid residue at position P₁′ being an acidic amino acid residue selected from the group consisting of a glutamic acid and an aspartic acid residue; the amino acid residue at position P₂′ being any amino acid residue except a leucine, a proline or a glycine residue, with the proviso that said peptide does not have the sequence as set forth in SEQ ID NO: 1;
detecting a product resulting of the PHEX enzymatic activity on said substrate; and wherein a difference in the amount of said product detected in the presence of said candidate compound as compared to that in the absence thereof is an indication that said candidate compound modulates PHEX.

11. A method as recited in claim 10, wherein said PHEX substrate further comprises a fluorophore unit capable of conferring fluorescence on said substrate, said fluorophore unit being attached to an amino acid residue at a first end of the peptide unit, and a quencher unit capable of providing intramolecular quenching of said fluorescence, said quencher unit being attached to an amino acid residue at a second end of the peptide unit, wherein P₁′ is located at least 2 amino acid residues distal to both the fluorophore unit and the quencher unit and wherein the product is detected through a modulation of fluorescence.

12. A method as recited in claim 10, wherein said amino acid residue at position P₁′ is aspartic acid.

13. A method as recited in claim 10, wherein said amino acid residue at position P₂′ is selected from the group consisting of a hydrophobic, an acidic and a polar amino acid residues.

14. A method as recited in claim 10, wherein said amino acid residue at position P₂′ is selected from the group consisting of an asparagine, a glutamine, a methionine, an alanine, a valine, a tryptophan, a threonine, a serine, a tyrosine, a phenylalanine, and an isoleucine residues.

15. A method as recited in claim 10, wherein said amino acid residue at position P₂′ is selected from the group consisting of a tryptophan, a threonine, a serine, a tyrosine, a phenylalanine and an isoleucine residues.

16. A method as recited in claim 10, wherein said amino acid residue at position P₂ is not an arginine, a lysine, an asparagine or a glutamine residue.

17. A method as recited in claim 10, wherein P₂-P₁-P₁′-P₂′ is as set forth in SEQ ID NO:2.

18. A method as recited in claim 11, wherein the fluorophore unit is Abz and the quencher unit is Dnp, and wherein Dnp is attached to a lysine residue.

19. A method as recited in claim 10, wherein the PHEX substrate has the chemical structure Abz-(SEQ ID NO:3)-Dnp.

20. A method as recited in claim 10, wherein the modulator is an inhibitor, and wherein a lower amount of said product detected in the presence of said candidate compound as compared to that in the absence thereof is an indication that said candidate compound inhibits PHEX.

21. A method for determining the presence and/or concentration of PHEX in a sample comprising
contacting said sample with a PHEX peptide substrate, said substrate including a peptide unit of at least 6 amino acids residues including a sequence P₂-P₁-P]′-P₂′ of 4 amino acid residues at positions P₂, P₁, Pi′ and P₂′ of the peptide unit, respectively; the amino acid residue at position P₂ being any amino acid residue; the amino acid residue at position P₁ being any amino acid residue except an isoleucine, a valine, or a histidine residue; the amino acid residue at position P₁′ being an acidic amino acid residue selected from the group consisting of a glutamic acid and an aspartic acid residue; the amino acid residue at position P₂′ being any amino acid residue except a leucine, a proline or a glycine residue, with the proviso that said peptide does not have the sequence as set forth in SEQ ID NO: 1;
assessing the presence and/or concentration of a product resulting of the PHEX enzymatic activity on said substrate; and
wherein the presence and/or concentration of said product can be correlated to the presence/concentration of PHEX in the sample.

22. A method as recited in claim 21, wherein said PHEX substrate further comprises a fluorophore unit capable of conferring fluorescence on said substrate, said fluorophore unit being attached to an amino acid residue at a first end of the peptide unit; and a quencher unit capable of providing intramolecular quenching of said fluorescence, said quencher unit being attached to an amino acid residue at a second end of the peptide unit, wherein P₁′ is located at least 2 amino acid residues distal to both the fluorophore unit and the quencher unit and wherein the product is detected through a modulation of fluorescence.

23. A method as recited in claim 21, wherein said amino acid residue at position P₁′ is aspartic acid.

24. A method as recited in claim 21, wherein said amino acid residue at position P₂′ is selected from the group consisting of a hydrophobic, an acidic and a polar amino acid residues.

25. A method as recited in claim 21, wherein said amino acid residue at position P₂′ is selected from the group consisting of an asparagine, a glutamine, a methionine, an alanine, a valine, a tryptophan, a threonine, a serine, a tyrosine, a phenylalanine, and an isoleucine residues.

26. A method as recited in claim 21, wherein said amino acid residue at position P₂′ is selected from the group consisting of a tryptophan, a threonine, a serine, a tyrosine, a phenylalanine and an isoleucine residues.

27. A method as recited in claim 21, wherein said amino acid residue at position P₂ is not an arginine, a lysine, an asparagine or a glutamine residue.

28. A method as recited in claim 21, wherein P₂-P₁-P₁′-P₂′ is as set forth in SEQ ID NO:2.

29. A method as recited in claim 22, wherein the fluorophore unit is Abz and the quencher unit is Dnp, and wherein Dnp is attached to a lysine residue.

30. A method as recited in claim 21, wherein the PHEX substrate has the chemical structure Abz-(SEQ ID NO:3)-Dnp.

31. A fluorogenic PHEX substrate comprising a peptide unit;
a fluorophore unit capable of conferring fluorescence on said substrate, said fluorophore being attached to an amino acid residue at a first end of the peptide unit; and
a quencher unit capable of providing intramolecular quenching of said fluorescence, said quencher being attached to an amino acid residue at a second end of the peptide unit;
the peptide unit comprising the sequence P₃-P₂-P₁-P₁′-P₂′-P₃′ of amino acid residues at positions P₃, P₂, P₁, P₁′, P₂′, and P₃′ of the peptide unit, respectively; the amino acid residue at position P₂ being any amino acid residue; the amino acid residue at position P₁ being any amino acid residue except an isoleucine, a valine, or a histidine residue; the amino acid residue at position P₁′ being an acidic amino acid residue selected from the group consisting of a glutamic acid residue and an aspartic acid residue; the amino acid residue at position P₂′being any amino acid residue except a leucine, a proline or a glycine residue, with the proviso that said peptide unit does not have the sequence as set forth in SEQ ID NO: 1.

32. A fluorogenic PHEX substrate as recited in claim 31, wherein said amino acid residue at position P₁′ is aspartic acid.

33. A fluorogenic PHEX substrate as recited in claim 31 wherein said amino acid residue at position P₂′ is selected from the group consisting of a hydrophobic, an acidic and a polar amino acid residues.

34. A fluorogenic PHEX substrate as recited in claim 31, wherein said amino acid residue at position P₂′ is selected from the group consisting of an asparagine, a glutamine, a methionine, an alanine, a valine, a tryptophan, a threonine, a serine, a tyrosine, a phenylalanine, and an isoleucine residues.

35. A fluorogenic PHEX substrate as recited in claim 31, wherein said amino acid residue at position P₂′ is selected from the group consisting of a tryptophan, a threonine, a serine, a tyrosine, a phenylalanine and an isoleucine residues.

36. A fluorogenic PHEX substrate as recited in claim 31, wherein said amino acid residue at position P₂ is not an arginine, a lysine, an asparagine or a glutamine residue.

37. A fluorogenic PHEX substrate as recited in claim 31, wherein said P2-P₁ -P′-P2′ is as set forth in SEQ ID NO:2.

38. A fluorogenic PHEX substrate as recited in claim 31, wherein the fluorophore unit is Abz and the quencher unit is Dnp, and wherein Dnp is attached to a lysine residue.

39. A method for identifying a PHEX modulator comprising contacting a candidate compound with PHEX in the presence of the fluorogenic PHEX substrate of claim 31; detecting a product resulting of the PHEX enzymatic activity on said substrate; and wherein a difference in the amount of said product detected in the presence of said candidate compound as compared to that in the absence thereof is an indication that said candidate compound modulates PHEX.

40. A method for determining the presence and/or concentration of PHEX in a sample comprising contacting said sample with the fluorogenic PHEX substrate of claim 31; detecting a product resulting of the PHEX enzymatic activity on said substrate; and wherein a difference in the amount of said product detected in the presence of said candidate compound as compared to that in the absence thereof is an indication that said candidate compound modulates PHEX.

41. A method as recited in claim 39, wherein said amino acid residue at position P₁′ is aspartic acid.

42. A method as recited in claim 39, wherein said amino acid residue at position P₂′ is selected from the group consisting of a hydrophobic, an acidic and a polar amino acid residues.

43. A method as recited in claim 39, wherein said amino acid residue at position P₂′ is selected from the group consisting of an asparagine, a glutamine, a methionine, an alanine, a valine, a tryptophan, a threonine, a serine, a tyrosine, a phenylalanine, and an isoleucine residues.

44. A method as recited in claim 39, wherein said amino acid residue at position P₂′ is selected from the group consisting of a tryptophan, a threonine, a serine, a tyrosine, a phenylalanine and an isoleucine residues.

45. A method as recited in claim 39, wherein said amino acid residue at position P₂ is not an arginine, a lysine, an asparagine or a glutamine residue.

46. A method as recited in claim 39, wherein P₂-P₁-P₁′-P₂′ is as set forth in SEQ ID NO:2.

47. A method as recited in claim 39, wherein the fluorophore unit is attached to P₃and the quencher unit is attached to P₃′.

48. A method as recited in claim 47 wherein the fluorophore unit is Abz, the quencher unit is Dnp, and P₃′ is a lysine residue.
